(19) United States
(12) Patent Application Publication
(10) Pub. No.: US 2015/0042360 A1
(43) Pub. Date: Feb. 12, 2015

(54) Method and Device for Person Identification

(71) Applicant: Robert Bosch GmbH, Stuttgart (DE)

(72) Inventors: Kathrin Graner, Fellbach (DE); Henning Hayn, Stuttgart (DE); Tatjana Thumm, Oberboihingen (DE)

(21) Appl. No.: 14/453,217

(22) Filed: Aug. 6, 2014

(30) Foreign Application Priority Data
Aug. 7, 2013 (DE) 10 2013 215 540.9
Mar. 28, 2014 (DE) 10 2014 205 838.4

(51) Int. Cl. A61B 5/117; A61B 5/053; G01N 33/483; G01R 27/02; G01N 27/02

(52) U.S. Cl. CPC A61B 5/117; G01R 27/02; G01N 27/02; G01N 33/4833; A61B 5/0531
USPC 324/630

(57) ABSTRACT

A method for person identification includes acquiring a data from impedance measurements on the body of a person and comparing the data with a reference data relating to the person. The method further includes inferring an identity of the person from the comparison. The method further includes carrying out the impedance measurements by a combination of at least one two-pole measurement and at least one four-pole measurement.

This application claims priority under 35 U.S.C. §119 to patent application no. DE 10 2013 215 540.9 filed on Aug. 7, 2013 in Germany and to patent application no. DE 10 2014 205 838.4 filed on Mar. 28, 2014 in Germany, the disclosures of which are incorporated herein by reference in its entirety.

### BACKGROUND

The present disclosure relates to a method and a device for person identification, data from impedance measurements on the body of a person being acquired.

An identification of persons plays an important role in many fields. For example, inputting person data or patient data is a prerequisite for many units for determining vital parameters or other person-specific parameters such as, for example, body weight, body water etc. for determining measured values. An unambiguous and reliable assignment between a patient and the respective measured values is important, particularly in telemedical applications. The patient data (for example name, age and body size) are usually input by hand before, for example, the patient is weighed, or before his/her blood pressure is measured, so that the appropriate measurement results can be passed on with a specification on the input name in the course of the telemedical application. However, no check is made in this case as to whether the measurements have actually been carried out with the person specified.

Various methods already exist for enabling the undertaking of non-manipulable person identification. For example, the fingerprint of a patient can be determined, an iris scan can be undertaken, a face recognition can be made by means of a camera and image processing, or the human voice can be examined and analyzed. However, said various methods generally require additional hardware and software, which gives rise to additional costs. Moreover, it is possible, for example, for face recognition by a camera to be felt as an intrusion into the private sphere of the person to be examined, which is also, where appropriate, not accepted against a legal background.

Other approaches to person identification are based on impedance measurements which are carried out on the body surface of the person or of the patient. Thus, for example, the international patent application WO 2000/016245 A1 describes a bioimpedance measurement for person identification, measurements being carried out on different segments of a body part. Here, biometric reference patterns for a body part are intercompared with instantaneous biometric patterns of said body part, and an identification is taken given an establishable similarity.

### SUMMARY

The disclosure relates to a method and a device for person identification, data from impedance measurements on the body of a person being acquired.

The disclosure provides a method for person identification which enables automatic identification of a person in a very simple and cost-effective way. In this case, data are acquired from impedance measurements on the body of the person. Said data are compared with reference data relating to the person and, if appropriate, the identity of the person is inferred therefrom. According to the disclosure, the impedance measurements are carried out by a combination of at least one two-pole measurement and at least one four-pole measurement. Two electrodes are used for the measurement. Four electrodes are used for the four-pole measurement. The core of the disclosure is a switch over between a two-pole and a four-pole measurement arrangement. It is possible by combining these two measurement principles to detect different parameters of the body composition which are very characteristic of a specific person, so that an unambiguous identification of the respective person can be derived from said measurement results. By contrast with conventional methods for person identification, which are based on a bioimpedance measurement, the method according to the disclosure has the great advantage that it is possible with only a small outlay of measurement and evaluation to acquire very characteristic data which permits an unambiguous assignment to one person. It is therefore possible without inputting additional information to identify the person or the patient by simple and cost-effective impedance measurements. On the basis of said automatic identification, it is possible, for example, to call up user profiles (name, age, sex, unit-specific settings etc.) from a unit store. For example, when further measurements (for example body analysis, vital parameters etc.) are carried out in the course of a telemedical application, said measurements can be carried out and evaluated on the called-up user profile.

In accordance with one embodiment according to the disclosure, it is expedient to carry out the impedance measurements for person identification with the same unit which is used for further measurements on the person or the patient. The unit can, for example, be provided for further impedance measurements in order to determine vital parameters or other person-specific parameters. For example, further measurements can be carried out in relation to the hydration state by means of impedance measurements, or other measurements can be provided, such as body weight measurements, for example. If the impedance measurements for person identification are carried out with the same unit, this has the particular advantage that the person identification requires no additional outlay. Rather, person identification can proceed in advance of the other measurements without the person or the patient needing to undertake additional actions therefor. It is particularly advantageous to have the method for person identification in accordance with the disclosure to be, for example, integrated in a conventional body analysis unit with which body constituents such as, for example, body water are determined by means of impedance measurements. No further outlay on apparatus is necessary in this refinement for carrying out the person identification according to the disclosure. Rather, the method according to the disclosure can be carried out straight away with the electrodes present in the body analysis unit and the remaining equipment. The method according to the disclosure permits the measurement technology already present in a body analysis unit to be used for the purpose of person identification by impedance measurements, thus enabling a simple, quick and, above all, unconscious checking of the patient data, and an unambiguous assignment of subsequent, recorded measurement values relating to the stored patient information. The method according to the disclosure is, for example, suitable for simple, uncomplicated application at home and is, in particular, suitable for use in the field of telemedicine. The measurements relating to the person identification can, for example, be carried out directly in advance of an actual body analysis measurement. In general, there is no need here for changes to the measurement positions, and so person identification can be carried out without this requiring the person or the patient to undergo further treatments. A further advantage of the method according to the disclosure for person identification on the basis of impedance measurements is that no invasive access to the patient is required. The electrodes required for the measurement are simply laid onto the body surface or, for example, stuck onto it without being attended by further-reaching drawbacks for the patient.

It is proposed that according to one embodiment of the disclosure, the method switches over between a two-pole and a four-pole measurement arrangement, and/or a combination of at least one two-pole measurement and at least one four-pole measurement. Different parameters of the body composition can be detected by combining these two measurement principles, so that it is possible to identify the respective person unambiguously. Conventional methods for person identification by means of impedance measurements generally require a very expensive evaluation of the acquired impedance data in order to enable an unambiguous assignment to one person. By contrast, the combination of two-pole measurements and four-pole measurements according to the disclosure records substantially more specific measured values which permit an unambiguous assignment to a specific patient straight away.

In one embodiment according to the disclosure, a tissue impedance is measured by the four-pole measurement, and the skin impedance is inferred from the values of the two-pole measurement through taking account of the measured tissue impedance. What is understood here by tissue is, in particular, muscle tissue, fat tissue, tissue fluid, fluid tissue (e.g. blood, lymph), functional tissue (organs) and other tissue types, which are to be found below the skin. Two electrodes are attached to the measurement sites for two-pole measurement. The measurement current is coupled in via these electrodes. Furthermore, the falling voltage is tapped via said electrodes. Included in the voltage drop in this case are electrode impedances, the skin impedance and the tissue impedance. That is to say, in the two-pole measurement the transition impedances of the electrodes and the skin impedance are also measured in addition to the tissue impedance. Four electrodes are attached to, or used at the measurement sites for four-pole measurement. The current is coupled in via the two outer electrodes. The falling voltage is tapped via the two inner electrodes. It holds to an approximation here that the measured impedance corresponds to the tissue impedance, this being so on the assumption that the input impedance of the measurement unit is greater than the tissue impedance, the electrode impedances and the skin impedance. Since this is generally the case of currently customary measurement units, this means that the electrode impedances and the skin impedance can be neglected so that the measured impedance corresponds approximately to the tissue impedance. The four-pole measurement is thus used, in particular, to measure the tissue impedance. The skin impedance can be inferred from the values of the two-pole measurement taking account of the measured tissue impedance. It follows that the skin impedance can be detected by settling the measurement results of the two measurement arrangements. Since, in particular, the skin impedance measurable in this way is a very characteristic value of a person, said measured values can be used for an unambiguous assignment to one specific person.

As a result of the embodiment according to the disclosure, the impedance measurements in the course of the method can permit a reliable recognition of a person presented for a measurement. Because of the different nature of the skin and of the body, it is also possible to observe here clear differences in the absolute values and in the characteristics of the impedances for individual persons. This is used according to the disclosure to identify persons, and to be able to call up appropriately assigned data from a unit memory. Said person identification further permits monitoring whether the details taken from a patient, for example in the course of a telemedical application, correspond to the facts, the result being to increase the reliability of measurements in the course of telemetric patient care.

It is further proposed that according to one embodiment of the disclosure, static impedance values at various frequencies can be determined in the impedance measurements for the person identification according to the disclosure. Furthermore, it is possible in addition or alternatively to determine the phase angle. A measurement of the phase angle is based on the fact that a cell membrane acts on the electric current like a capacitor which is charged given an increase in the voltage present, and discharged given a decrease in the voltage present. A time shift occurs in the AC circuit as a result of said capacitive properties of the cell membrane. The current maximum leads the voltage maximum. Since a sinusoidal alternating current signal is involved, the shift can be measured in degrees and be denoted as a phase shift. The measurement of the shift is performed by comparing the input signal with the output signal. Furthermore, it is possible to compare the times at which the maximum in the signal respectively occurs. Said measurement results can be used to acquire further, very individual-specific data which can be employed for the person identification according to the disclosure. Alternatively or in addition, the dynamic response of the body to the incoming current can be measured, in particular a step current being impressed and the so called “step response” of the body being detected. It is possible by means of said various methods of measurement and evaluation to obtain very detailed information or measurement results which permits an unambiguous person identification.

As a result of the embodiment according to the disclosure, after an identification of the respective person and/or the respective patient, it is possible, by way of example, to call up automatically a stored user profile on which further analyses are based.

It is conceivable, according to one embodiment, that the impedance measurements can, for example, be undertaken on the foot soles and/or on the inner hand surfaces and/or on the outer circumference of an extremity, for example on a wrist joint and/or on an ankle joint. In principle, this can be done by using body analysis units known per se which have, for example, appropriate support devices for the hands and/or the feet and are equipped with appropriate electrodes. The electrodes can, for example, also be applied to the body in the form of cuffs, for example cuffs for the wrist and/or ankles. The body analysis units must be set up for the disclosure so that the electrodes can be switched for two-pole measurement and for four-pole measurement.

It is also proposed according to one embodiment that the electrodes for the two-pole measurements and the four-pole measurements can be interconnected in a different way during measurement on the outer circumference of an extremity. By way of example, for the measurements it is possible to interconnect electrodes on one side of the extremity and/or electrodes on sides of the extremity approximately opposite one another. Very person-specific data which are suitable for an unambiguous person identification can be obtained to the measurement results thereby achievable.

According to one embodiment of the method, the four-pole measurement is carried out as a segmental bioimpedance measurement, impedances of individual body segments, for example the right arm, the left arm, the torso, the right leg and/or the left leg, being determined. Consequently, the segmental bioimpedance measurement can be used to detect various characteristic values of a patient which, when seen together, permit a particularly reliable identification of the person.

In addition, it is proposed that according to one embodiment, the four-pole impedance measurement on an arm, for example, can be undertaken so that two electrodes are used in the region of the hand, and two electrodes in the region of the shoulder. For example, adhesive electrode can be used for this. However, even small changes in the electrode positions can be enough to affect the measurement result. In a particularly preferred refinement, the segmental bioimpedance measurement is performed in such a way that electrodes are attached to the body and/or are brought into contact with the body in such a way that the current path and the measurement path overlap only in the body segment to be measured. By way of example, for this purpose the electrodes can be brought into contact with the body in the region of the hands and in the region of the feet, for example by integrating the electrodes in the handles and the treads of a body analysis unit. By switching the electrodes appropriately, the impedance measurements of individual extremities and/or on the torso can be undertaken consecutively in the case of such an arrangement without the need to undertake further adaptations.

As a result of one embodiment of the method according to the disclosure, the measured data, for example the impedance Z and the phase angle φ, of two or more body segments are recorded in a prescribable frequency range. In a preferred way, it is as large a frequency range as possible, for example from 1 to 100 kHz or from 1 to 1000 kHz, that is used for this purpose. Measurement curves can be derived from said measured data. With the aid of characteristic features of said measurement curves, for example the extreme points, it is possible to perform an evaluation, appropriate characteristic features being compared with reference values which have been determined in the same way in advance and stored, so that said comparison can be used to make an assignment to a specific person.

According to one embodiment of the method, during the evaluation of the data of the impedance measurements account is taken of further person-specific measured values. For example, weight data and/or electrocardiogram data which further facilitate person identification can contribute as further body features.

Once a person identification is done, a computationally stored user profile can preferably be called up. This is helpful, in particular, for those applications in which the person identification is preceded by a further examination of the respective person, for example in the course of telemedical applications. For example, the method for person identification according to the disclosure can be coupled with further impedance measurements for a body analysis. In this case, the person identification is advantageously temporally coupled to the impedance measurements for the body analysis.

It is proposed that in one embodiment according to the disclosure can comprise the use of a body analysis unit for carrying out the person identification in accordance with the described method. Conventional body analysis units already use bioimpedance measurements to analyze the body composition, for example the total body water content and the muscle mass and fat mass, the different conductivities of various body constituents being determined. A precondition for the use of a conventional body analysis unit on the basis of bioimpedance measurements for the method for person identification according to the disclosure is that the electrodes of the body analysis unit are set up such that they can be switched in such a way that two-pole measurements and four-pole measurements can be carried out.

In addition, it is proposed that one embodiment further comprises a device for person identification by impedance measurements, the device comprising at least four, preferably at least eight, electrodes for making contact with the body surface of a person. Furthermore, a current source is provided for supplying the electrodes with current. Said device is characterized in that the electrodes can be switched over for both two-pole measurements and for four-pole measurements. By way of example, the device is, furthermore, set up for a body analysis on the basis of impedance measurements. Said device according to the disclosure can be used with particular advantage above all in the course of telemedical applications. By way of example, the person identification according to the disclosure which can be carried out hereby can automatically call up a specific user profile so that further measurements of vital parameters or other person-specific parameters can be unambiguously assigned to the person.

Finally, it is conceivable that according to one embodiment, the device comprises a computer program as well as a computer program product with program code which is stored on a machine-readable carrier. Said computer program or the computer program product is provided to carry out a method for person identification when the computer program is executed on an arithmetic logic unit or a control unit. By implementing the method according to the disclosure in a computer program, it is possible to make use of the advantages of the person identification according to the disclosure, for example even in conventional body analysis units which operate on the basis of bioimpedance measurements, when said body analysis units are set up for a combination of two-pole measurements and four-pole measurements.

### BRIEF DESCRIPTION OF THE DRAWINGS

Further advantages will become clear from the following description of the drawings. Exemplary embodiments of the disclosure are illustrated in the drawings. The drawings, the description and the claims contain numerous features in combination. A person skilled in the art will also consider the features individually as appropriate and will combine them to form further expedient combinations.

In the drawings:

FIG. 1A is a schematic of a two-pole measurement according to the disclosure;

FIG. 1B is a schematic of a two-pole measurement according to the disclosure;

FIG. 2A is a schematic of a four-pole measurement according to the disclosure;

FIG. 2B is a schematic of a four-pole measurement according to the disclosure;

FIG. 3 is a schematic of an exemplary measurement setup for carrying out the method according to the disclosure;

FIG. 4 is a schematic of a measurement arrangement having a sleeve with electrodes for carrying out the method according to the disclosure;

FIG. 5 shows a circuit diagram for impedance measurements in accordance with the method according to the disclosure;

FIG. 6 shows a circuit diagram for switching over between a two-pole circuit and a four-pole circuit by means of short circuiting two electrodes according to the disclosure;

FIG. 7 shows a circuit diagram for switching over between a two-pole circuit and a four-pole circuit by excluding two electrodes according to the disclosure;

FIG. 8 is a schematic of an example for arranging electrodes for a segmental impedance measurement according to the disclosure;

FIG. 9 is a schematic of a further example for arranging electrodes for a segmental impedance measurement according to the disclosure; and

FIG. 10 shows an example of an evaluation of the method according to the disclosure with the aid of locus curves of measurements on a right arm according to the disclosure.

### DETAILED DESCRIPTION

The present disclosure is based on the fact that humans differ in their physiological composition and therefore also in their electrical properties. Thus, for example, between individual persons there are clear differences between the compositions of the skin on the foot soles or on the inner hand surfaces. The composition of muscles and blood vessels likewise differs between individuals. According to the disclosure, measurement technology is used to detect said differences and evaluate them in order to be able to identify persons by impedance measurements. For example, users can thus be identified by measurement units, for example by body analysis units. The method for person identification according to the disclosure can therefore, for example, be integrated in a body analysis unit. Firstly, a first assignment to a person is undertaken by a reference measurement, the measured data from the impedance measurements being assigned to the respective person. It is also possible thereby to assign the person further measured data such as, for example, measured data relating to body weight or to an electrocardiogram. By way of example, name, sex, age and body size of the person are stored for the assignment. As the person identification according to the disclosure is being carried out, the data of the impedance measurements are respectively recorded anew and, in particular, automatically compared with the reference values in the unit memory. Assignment to the appropriate person can be performed in this way.

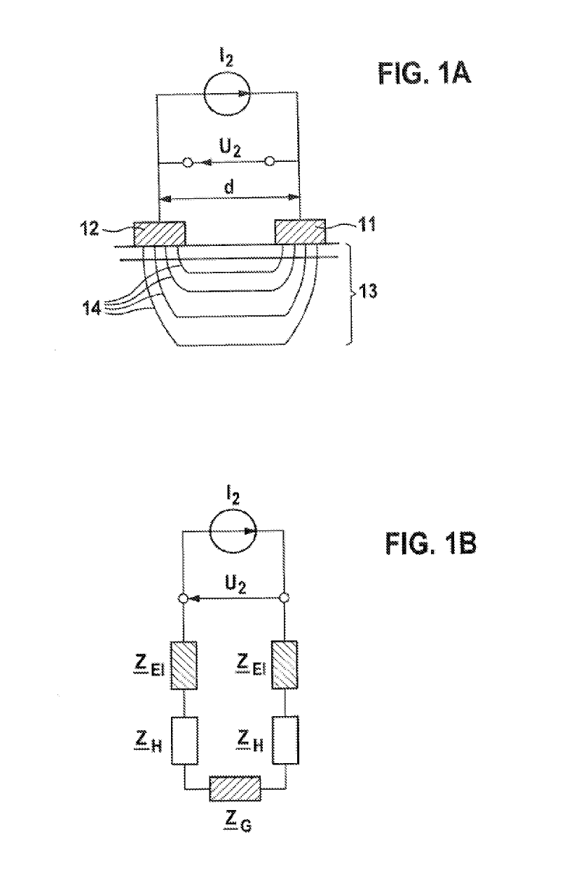

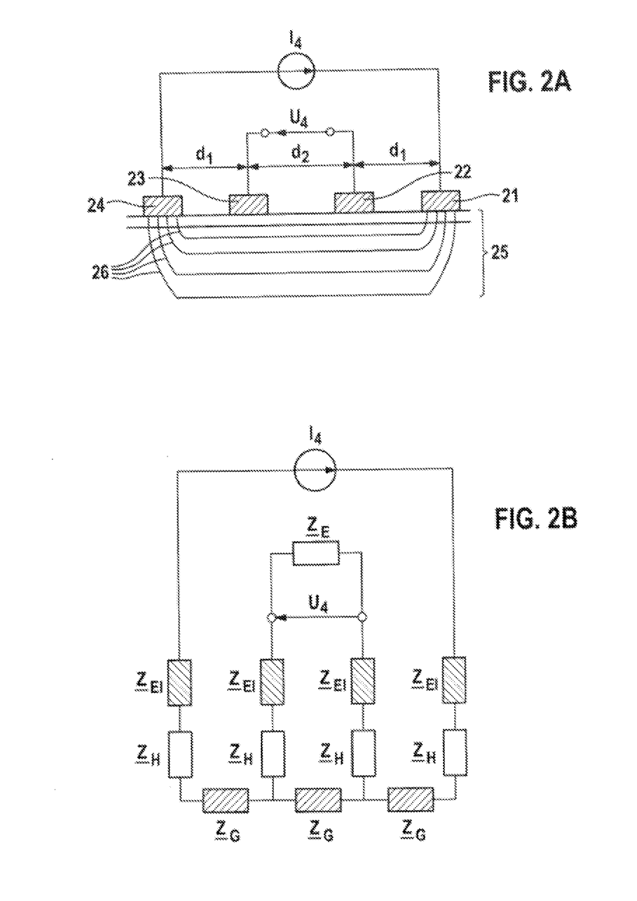

FIGS. 1 and 2 firstly illustrate the principles of two-pole measurement and four-pole measurement by means of which the impedance measurements are carried out on the body surface of a person in order to undertake a person identification. According to the disclosure, said two measurement principles are combined with one another and appropriately evaluated in order to obtain characteristic data relating to the person.

The principle of two-pole measurement is illustrated in FIG. 1. Here, two electrodes 11 and 12 (FIG. 1A) are attached to the measurement sites on the body. The measurement current (I₂) is coupled into the object via the electrodes 11 and 12. Furthermore, the voltage (U₂) falling across the object is tapped via the electrodes 11 and 12. The skin 13 on which the electrodes 11 and 12 rest is subdivided diagrammatically into two layers through which the current lines 14 run. FIG. 1B shows an equivalent circuit diagram for said arrangement. The AC resistances (impedances Z) for the electrodes are denoted by Z_{El}. The impedances for the skin are denoted by Z_{H}. The tissue impedance is denoted by Z_{G}. In accordance with Ohm's law, the impedance (Z₂) lying between the two electrodes 11 and 12 may be calculated in accordance with:

$$\text{?} = \text{?} + 2 \text{?} + \text{?} = \frac{\text{?}}{\text{?}}$$

$$\text{?} \text{indicates text missing or illegible when filed}$$

Account is taken in this way of all the impedances which lie on the path of the current from one electrode to the other. That is to say, in two-pole measurement said impedances are, in particular, the first electrode impedance (Z_{El}), the skin impedance (Z_{H}), the tissue impedance (Z_{G}), the skin impedance again and, finally, the impedance of the second electrode. Consequently, during two-pole measurement, in addition to the tissue impedance the transition impedances of the electrodes and the skin impedance are also measured. For two-pole measurement, it is possible, in particular, to use electrodes with a relatively low transition impedance in the course of the method according to the disclosure, for example electrodes having the largest possible surface area. Alternatively or additionally, it is possible to use materials which are particularly good conductors such as, for example, silver chloride or black ruthenium, in order thus to keep the electrode impedance low and thereby increase the measurement resolution.

In a comparable way, FIG. 2 illustrates a four-pole measurement in which four electrodes 21, 22, 23 and 24 (FIG. 2A) are used at the measurement sites. The current (I₄) is coupled into the measurement object via the two outer electrodes 21 and 24. The falling voltage (U₄) is tapped via the two inner electrodes 22 and 23. The current running through the skin 25 is indicated by the current lines 26. FIG. 2B is an equivalent circuit diagram, the various AC resistances at the electrodes, the skin and the tissue being designated correspondingly as impedances as in FIG. 1B. This results in:

$$\frac{{\underline{U}}_{4}}{{\underline{I}}_{4}} = \underline{Z} = \frac{{\underline{Z}}_{G}}{1 + \frac{{\underline{Z}}_{G}}{\text{?}} + 2 \frac{\text{?}}{\text{?}} + 2 \frac{\text{?}}{\text{?}}} \approx {\underline{Z}}_{G}$$

$$\text{?} \text{indicates text missing or illegible when filed}$$

by setting up the current and voltage ratios at the individual impedances to be taken into account. Here, the measured impedance corresponds approximately to the tissue impedance, this being the case under the assumption that the input impedance Z_{E} of the measurement unit is very much larger than the impedances Z_{G}, Z_{El} and Z_{H}. This is done by conventional measurement units, as a rule. Consequently, the electrode impedances and the skin impedance can be neglected. By contrast of a two-pole measurement, the measurement resolution of a four-pole measurement therefore can be generally higher. The method according to the disclosure combines two-pole and four-pole measurement so that the different resolution associated and measurement ranges with said measurement principles are used and can contribute to the acquisition of individual-specific data. By computing the various measured data, it is possible, in particular, to acquire the skin impedance, which is very characteristic of an individual.

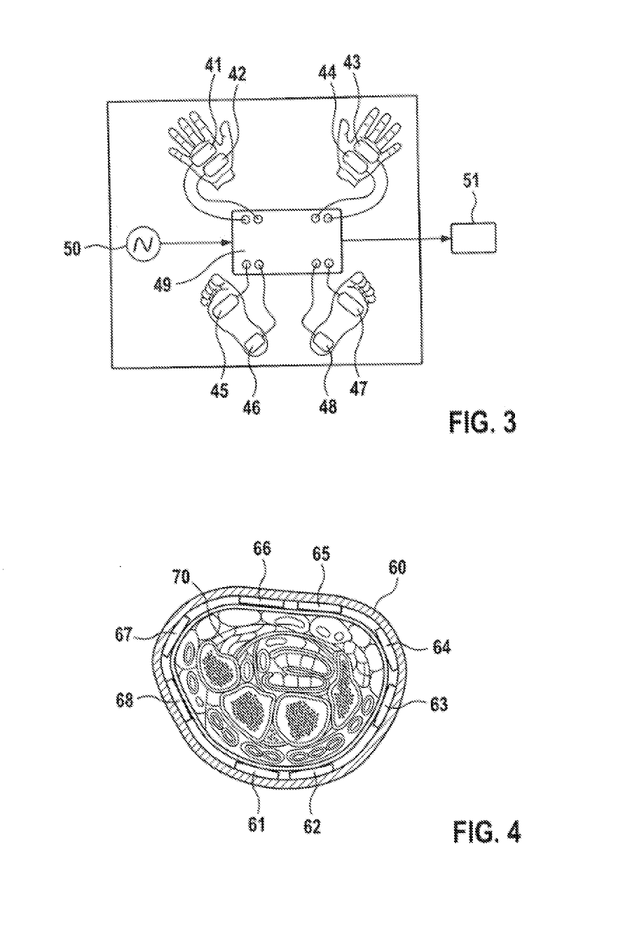

FIG. 3 illustrates schematically a possible setup for carrying out the method for person identification according to the disclosure. In said refinement, eight electrodes 41 to 48 are provided of which two each are attached to two hand surfaces and to two foot soles of a patient. These can be adhesive electrodes or other dry, nonadhesive electrodes. Furthermore, the electrodes can also be integrated in handles and/or standing surfaces of a measurement unit. Preferably, sinusoidal are used alternating currents of various frequencies for measurement. According to the disclosure, an alternation between two-pole and four-pole measurement is carried out. Consequently, the measurement unit is designed so that in the case of two-pole measurement only the two electrodes required for the measurement are actuated, while the other electrodes are deactuated, that is to say are devoid of current. Said deactivation is canceled for the four-pole measurement. It is, furthermore, advantageous when the two-pole measurements and the four-pole measurements are carried out at various body sites, that is to say with various electrodes, for example. It is therefore expediently possible for each electrode to be both deactuated and actuated. For example, it is possible to measure the only two electrodes on the left hand surface or, by way of example, to actuate respectively one electrode on each extremity in a four-pole measurement. The actuation of the individual electrodes is performed via the circuit 49 and by means of the current source 50. The frequencies applied are variable in this case, and are set as a function of the respective parameters to be measured. The measurement results are processed by suitable algorithms in a data processing unit 51 and evaluated. In particular, during the evaluation a comparison is made with the data of reference measurements, that is to say the measured data are matched with the data which are stored for a specific person so that the person identification can be performed in this way.

Measurements on the foot soles are particularly preferred. The electrodes 45 and 46 and, correspondingly, 47 and 48 which are provided for measurement on the foot soles are preferably placed such that there is enough space between them that no directly conducting contact can exist. The measurements can be carried out respectively at an electrode pair of a foot so that the skin impedance can firstly be determined by this arrangement. The measurement values differ between individuals because people have various skin states such as, for example, dry or greasy skin, callosity or scars. For example, dry skin and callosity lead to higher impedance values by comparison with creamed or wet skin. Since these are features that do not change continuously as a rule, a first assignment of the impedance values to a specific person is already possible in this way. Equally, the measurement can be carried out on the hand inner surfaces. In this case, the measured values generally vary more strongly for a person than on the feet hence, for example, hand creams or continual hand washing influence the impedance values.

The disclosure provides a combination of two-pole and four-pole measurements in order to achieve a particularly reliable person identification on the basis of the measured impedance values. Thus, in addition to information related to the nature of the skin from the two-pole measurement described, there is also obtained from the two-pole measurement described further information relating to the composition of the body from the four-pole measurement, in particular further information relating to proportion of muscle, water, fat and bone, which permits an unambiguous person identification. Regarding the combination of two-pole measurements and four-pole measurements, all the influences of the substances lying between the electrodes are measured in principle. For example, the impression of the muscles, the thickness of the fat layer and the properties of the bones feature in the measurement results in this case. Depending on whether the respective person is, for example, a stout or a sporty person, clear differences are to be expected in the level of the impedance values and in the curves of the impedance values as a function of the measurement frequency. For example, the impedance value rises given a falling water and muscle proportion as well as given an increased fat proportion.

The electrodes 41 to 48 can be differently connected for the combined two-pole measurement and four-pole measurement. In principle, any combination of two and four electrodes is possible. The following electrode combinations are preferred in order to obtain particularly informative measured values:

| Whole body four-pole | Whole body two-pole |
|---|---|
| measurement: | measurement: |
| −41, 42, 47, 48 | −41, 47 |
| −43, 44, 45, 46 | −42, 48 |
| −45, 46, 47, 48 | −43, 45 |
| −47, 48, 41, 42 | −44, 46 |

| Four-pole measurement | Partial two-pole |
|---|---|
| on the upper body: | measurement: |
| −41, 42, 43, 44 | −41, 42 |
| | −43, 44 |
| | −45, 46 |
| | −47, 48 |

In a further embodiment of the disclosure, a cuff is used for the measurement which can, for example, be attached to the wrist or an ankle of a person. FIG. 4 shows schematically a possible embodiment of such a cuff 60 in cross section. In this example, the cuff 60 has eight electrodes 61 to 68. What is shown is the section through a wrist 70, around which the cuff 60 is laid.

There are various possibilities for interconnecting the electrodes 61 to 68. Firstly, measurement can be performed either through the wrist 70 or with electrodes on only one side of the wrist. In this case, all the influences of the substances lying between the electrodes are measured. Various measurement paths, and thus various substances and compositions, can be detected and evaluated by a combination of two-pole measurements and four-pole measurements in order to obtain individual data for a person which are to be assigned unambiguously. The impedance values thus obtained can be used to evaluate and identify either individually or, for example, as calculated mean values. In particular, the measured data relating to skin-specific properties which can be acquired in this way are very informative parameters for person identification. The following combinations for switching electrodes return particularly informative measured values:

| Four-pole measurement on | Two-pole measurement on |
|---|---|
| one side of the wrist: | one side of the wrist: |
| −61, 62, 63, 64 | −61, 62 |
| −63, 64, 65, 66 | −63, 64 |
| −65, 66, 67, 68 | −65, 66 |
| −67, 68, 61, 62 | −67, 68 |

| Four-pole measurement | Two-pole measurement |
|---|---|
| through the wrist: | through the wrist: |
| −61, 62, 65, 66 | −61, 66 |
| −63, 64, 67, 68 | −62, 65 |
| | −63, 67 |
| | −64, 68 |

The impedance measurements according to the disclosure can be further supplemented by measuring a so-called step response in which the dynamic response of the human body to the incoming current. In this case, a current step, in particular a ramp current or triangular current, is coupled into the body via the electrodes. A step response of the voltage U falling in the body can be measured thereby. Moreover, the resulting impedance

$$\underline{Z} = \frac{U}{I}$$

can also be calculated by Ohm's law. Information relating to the dynamic response of the body is obtained in this way. This response is also a function of the composition of the body under inspection and will change depending on the fat, muscle, bone and skin proportions present, and so it is possible to distinguish between persons in this way. Said measurements can be carried out both by a four-pole measurement and by a two-pole measurement. It is particularly preferred to combine said various measurement methods with one another so that the lack of ambiguity of the measurement results with regard to person identification is yet further improved.

In a further variant of the method for person identification according to the disclosure, the measured values obtained by the impedance measurements are supplemented by further person-specific parameters in order to enable a more reliable assignment of the values to a person. Said supplementary parameters can be, for example, the body weight of the person to be examined and/or electrocardiogram data. In particular, the electrocardiogram signals show very characteristic curves which enable an assignment to one specific person. The greater number of parameters that can be used to identify the person, the more accurately can an assignment be done, and it is to be taken into account in this case that the use of further parameters can in some circumstances lengthens the measurement time required.

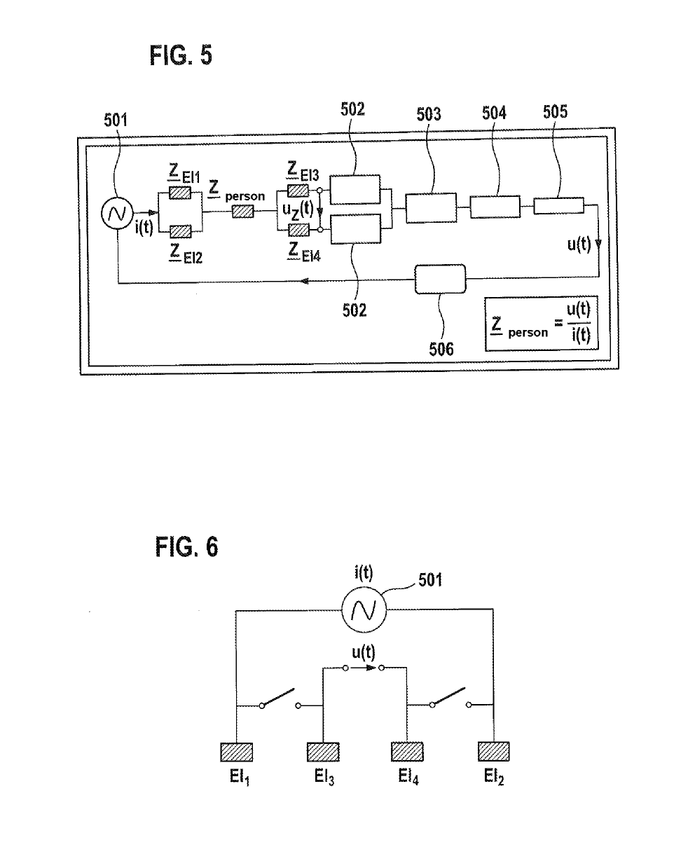

FIG. 5 shows a general circuit diagram for the impedance measurements in the course of the method according to the disclosure. A current source 501, in particular an AC generator, is used to supply the various electrodes with variable measurement frequencies. The control units 506 control the measurement cycle. Four different electrodes are actuated in the case of four-pole measurements. It holds in this case that: Z_{El1}≠E_{El2}≠E_{El3}≠E_{D4}. If these measurements are made in a two-pole arrangement, the voltage is tapped via the electrode pair via which the current is also fed into the body. It can therefore be understood from the circuit diagram in FIG. 5 that Z_{El3}=Z_{El1} and Z_{El4}=Z_{El2}. The measurable impedances are denoted in general as Zₚₑᵣₛₒₙ. Furthermore, use is made in the circuit of an impedance converter 502, a differential amplifier 503, a further amplifier 504 and an A/D converter 505. The results of the impedance measurement, which represent the ratio of the measurable voltage as a function of the imposed alternating current, can be processed and evaluated in the control unit 506, for example a computer, a microcontroller unit or, generally, a control device. Data processing can also be performed externally.

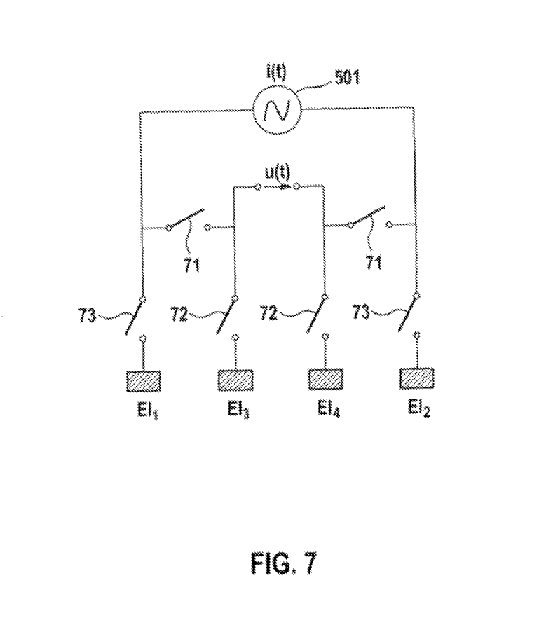

It is essential for the device according to the disclosure to switch over between two-pole measurements and four-pole measurements. Exemplary circuit diagrams for possible switchovers are illustrated in FIGS. 6 and 7. Switching over by means of a short circuit between two electrodes in each case is illustrated in FIG. 6. If the two switches are open, four electrodes are used to measure. Given closed switches, the electrode El₁ is short-circuited to the electrode El₃, and the electrode El₂ is short-circuited with the electrode El₄, the result being that the measurement be done by two electrodes. However, said electrodes have double the surface area of the original measurement electrodes. If such a change in surface area is not desired, it is possible, for example, to measure a variant of the switchover as illustrated in FIG. 7. In said variant, the electrodes are not short-circuited, but two further switch pairs are used to exclude two electrodes from the measurement circuit. The switch pair 71 is opened for the four-pole measurement. The switch pair 72 and 73 are closed. For a two-pole measurement with the two outer electrodes El₁ and El₂, the switch pairs 71 and 73 are closed while the switch pair 72 remains open. The connection to the two inner electrodes El₃ and El₄ is thereby interrupted. If, by contrast, the aim is to measure two inner electrodes El₃ and El₄, the switch pairs 71 and 72 are closed while the switch pair 73 remains open. The connection to the electrodes El₁ and El₂ is thereby interrupted.

The switches are preferably switches that can be electronically actuated, such as transistors or relays which can be actuated automatically by the measurement software used on a computer or a microcontroller.

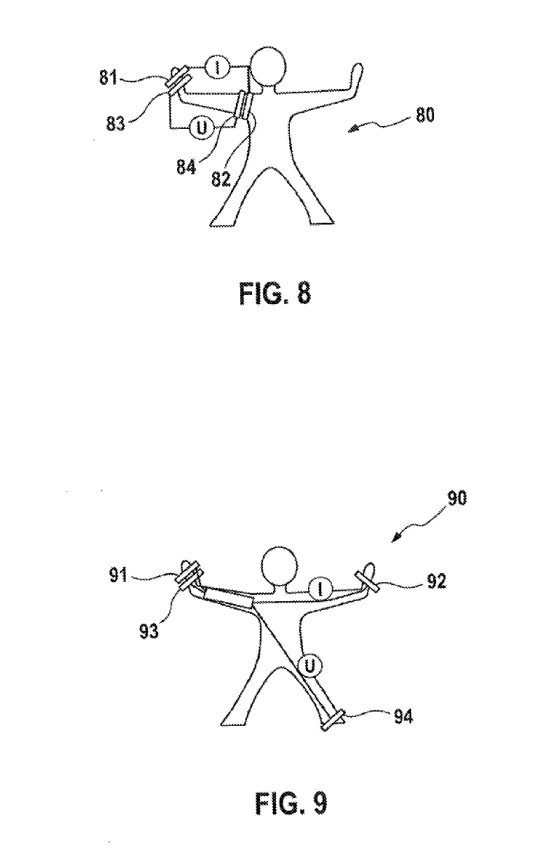

FIG. 8 illustrates one possibility of how the electrodes can be placed for a four-pole measurement. In this case, the bioimpedance on one arm is measured, it being possible to carry out said measurement in an appropriate way, for example on the other body extremities. Said arrangement is suitable, in particular, for segmental bioimpedance measurement. Four electrodes are attached to an arm of the person 80, it being possible, for example, for the electrodes to be adhesive. Two outer electrodes 81 and 82 are situated in the region of the hand or in the region of the shoulder. Two inner electrodes 83 and 84 are likewise situated in the region of the hand or the shoulder. A current is coupled in via the outer electrodes 81 and 82. The resulting voltage is tapped via the inner electrodes 83 and 84. Particular electrode positions can, however, affect the measurement result, since the length of the distance between the electrodes can influence the impedance values. When the extremities, that is to say arms and legs, of a person are considered approximately as cylindrical conductors, the length 1, that is to say the distance between the electrodes, affect the impedance Z in accordance with the following equation:

$$Z = ρ \cdot \frac{l}{A} ,$$

p being the specific resistance of the conductor, l the length thereof and A the cross-sectional area thereof. It is therefore advantageous for the reliability of the measured values when the correct placing of the electrodes is performed with care.

FIG. 9 illustrates a further possibility for positioning the electrodes, in particular for the segmental bioimpedance measurement. A person 90 is illustrated. The electrodes 91, 92 and 93 are positioned in the region of the hands. The fourth electrode 94 is positioned in the region of a foot. The current is coupled in via the electrodes 91 and 92. The resulting voltage is tapped via the electrodes 93 and 94. The electrodes are positioned in this case on the body so that the current path and the measurement path overlap only in the body segment to be measured, that is to say in an arm, in this case.

Here, the electrodes can be stuck onto the skin. It is preferred, in particular, to integrate the electrodes in a body analysis unit. In particular, the electrodes can be accommodated in the handles and the treads of such a unit. Segmental impedance measurement can be carried out without further outlay in this way by connecting the electrodes appropriately.

Segmental impedance measurement can be carried out with particular advantage in the course of the method according to the disclosure, since a particularly reliable identification of a specific person can be measured thereby. When the electrodes are integrated in a body analysis unit, there is no need for additional measures to carry out the method according to the disclosure, in particular no electrodes need be stuck onto the body. Once identification is done, it is possible, by way of example, to call up a stored user profile in the body analysis unit.

By way of example, it is possible to carry out and evaluate segmental impedance measurement by firstly recording measured data, in particular the impedance Z and the phase angle φ, of some or all body segments of a person in a prescribable frequency range, for example between 1 and 100 kHz or, for example between 1 and 1000 kHz. In addition to the curves of the raw data as a function of the measurement frequencies (Z(f) and φ(f)), the locus curves of the measurement are advantageously also considered. For this purpose, the imaginary part (Im) and the real part (Re) can be calculated and plotted as locus curves from the raw data:

Im=Z·sin(φ)

Re=Z·cos(φ).

The measurement curves can be used to identify characteristic features that can be used to describe the curves and distinguished them from another. For example, the coordinates of the high points in the course of the curves, or the polynomials which approximate the course of the curves, or the difference between the maximum and the minimum real part can be considered and analyzed.

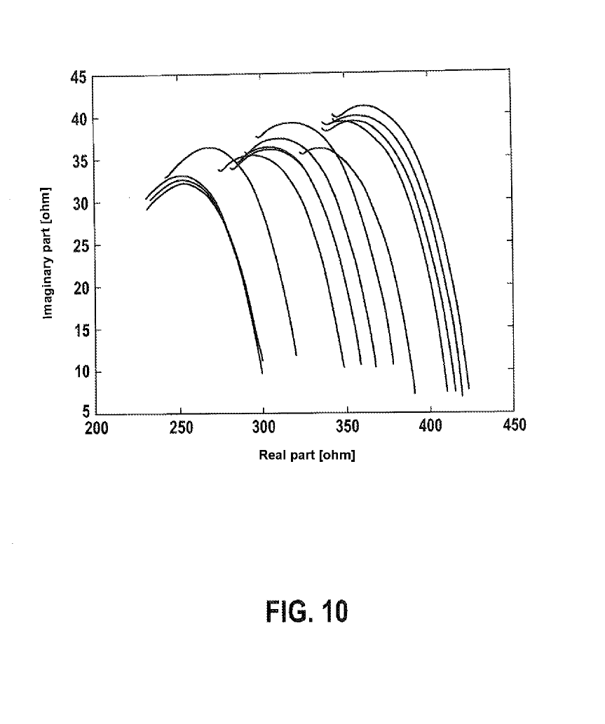

FIG. 10 shows exemplary locus curves, the imaginary part is plotted above the real part, in ohms respectively. Said various locus curves were recorded on a right arm. The measurement curves, or the characteristic features of the respective curves can be compared with stored reference data. For persons under consideration, measured data for corresponding reference values are measured in advance and analyzed, and characteristic values are stored. The measured data to be assigned are likewise characterized by calculating the features so that said features can be compared with the corresponding points or data of the reference data. The assignment to a person stored in the unit can be done when the values or data agree.

What is claimed is:

1. A method for person identification, comprising:
acquiring data from impedance measurements on the body of a person;
comparing the data with reference data relating to the person; and
inferring an identity of the person therefrom,
wherein the impedance measurements are a combination of at least one two-pole measurement and at least one four-pole measurement.

2. The method according to claim 1, further comprising:
inferring a skin impedance from the at least one two-pole measurement and the at least one four-pole measurement, wherein the at least one four-pole measurement is a tissue impedance.

3. The method according to claim 2, wherein the at least one four-pole measurement and the at least one two-pole measurement are at least one of (i) static impedance values of various frequencies, (ii) a phase angle, and (iii) a dynamic response of the body to an incoming current.

4. The method according to claim 1, wherein the impedance measurements are undertaken on at least one of foot soles, inner hand surfaces, and an outer circumference of an extremity.

5. The method according to claim 4, wherein the impedance measurements are undertaken on the outer circumference of the extremity, the at least one two-pole measurement and the at least one four-pole measurement are measured by at least one of (i) electrodes on one side of the extremity; and (ii) electrodes on sides of the extremity approximately opposite one another.

6. The method according to claim 4, wherein the at least one four-pole measurement is at least one segmental impedance measurement in which impedances of individual body segments are determined.

7. The method according to claim 6, wherein electrodes for the impedance measurements are attached to the body so that a current path and a measurement path overlap only in a body segment to be measured.

8. The method according to claim 6, wherein the at least one segmental impedance measurement is recorded from at least two body segments in a prescribable frequency range, and an evaluation is performed with the aid of characteristic features of measurement curves derivable therefrom.

9. The method according to claim 8, wherein during the evaluation, account is taken of further measured values relating to body features.

10. The method according to claim 9, further comprising calling up a computationally stored user profile after person identification is performed.

11. The method according to claim 10, wherein the method for person identification is temporally coupled to impedance measurements for a body analysis.

12. The method according to claim 1, wherein the method is carried out using a body analysis unit.

13. The method according to claim 1, wherein the impedance measurements are undertaken on at least one of a wrist joint and an ankle joint.

14. A device for person identification by impedance measurements, comprising:
at least four electrodes configured to make contact with a body surface of the person, and
a current source configured to supply the at least four electrodes with current,
wherein the at least four electrodes are configured to be switched between at least one two-pole measurement and at least one four-pole measurement.

15. A device according to claim 14, wherein the device is configured to provide a body analysis on the basis of the impedance measurements.

16. A device according to claim 14, wherein the device is configured for use in a telemedical application.

17. The device according to claim 14, wherein the at least four electrodes are at least eight electrodes.

18. A computer-readable storage medium having instructions stored which, when executed by an arithmetic logic unit or a control unit of a processor, causes the processor to perform operations comprising:
acquiring data from impedance measurements on the body of a person;
comparing the data with reference data relating to the person; and
inferring an identity of the person therefrom,
wherein the impedance measurements are a combination of at least one two-pole measurement and at least one four-pole measurement.
